(19) United States
(12) Patent Application Publication
(10) Pub. No.: US 2026/0111980 A1
(43) Pub. Date: Apr. 23, 2026

(54) LEARNING PROPENSITY INFORMATION DETERMINATION SYSTEM BASED ON GENETIC TEST INFORMATION, AND LEARNING PROPENSITY INFORMATION DETERMINATION METHOD BASED ON GENETIC TEST INFORMATION

(71) Applicant: GENE STORY KOREA INC., Seoul (KR)

(72) Inventor: Seung Hwan YU, Seoul (KR)

(21) Appl. No.: 19/122,271

(22) PCT Filed: Oct. 24, 2023
(86) PCT No.: PCT/KR2023/016578

§ 371 Date: Apr. 18, 2025

(30) Foreign Application Priority Data
Oct. 28, 2022 (KR) 10-2022-0141231

(51) Int. Cl. G06Q 50/20; A61B 5/16

(52) U.S. Cl. CPC G06Q 50/205; A61B 5/16

(57) ABSTRACT

A learning propensity information determination system based on genetic test information, according to one embodiment, comprises: a basic information generation unit for generating basic information by receiving information about a testee; a genetic test information generation unit for generating genetic information by analyzing a genetic sample of the testee; and a learning propensity information determination unit for generating cognitive category learning propensity information on the basis of the genetic information, generating non-cognitive category learning propensity information on the basis of the genetic information and the basic information, and generating learning propensity information by integrating the cognitive category learning propensity information and the non-cognitive category learning propensity information.

### TECHNICAL FIELD

The present invention relates to a system and method for determining learning tendency information on the basis of genetic test information, and more particularly, to a system for determining learning tendency information that determines learning tendencies on the basis of genetic test information of a subject and recommends an academic direction to improve the subject's academic achievement, and a method of determining learning tendency information on the basis of genetic test information using the system.

### BACKGROUND ART

There are various personality and aptitude testing methods to identify personalities and aptitudes for the education of children and adolescents or the retraining of adults. Most existing personality and aptitude testing methods involve conducting questionnaire tests for a certain period of time using questionnaires that are prepared in advance.

The results of these personality and aptitude tests are highly likely to change depending on a mental environment in which given problems have to be solved for a certain period of time, and may vary depending on the will, knowledge, temporal and spatial environments, and emotions of an individual subject. Also, since the test results are quantified or categorized for interpretation, there are limitations in accurately identifying characteristics that only the individual has.

In addition, there are also limitations in identifying traits such as behavior, emotions, perceptions, judgments, and the like that occur at an unconscious level.

To overcome these limitations, a system for providing a solution using a DNA aptitude test is being developed, but this is a system that simply categorizes the results of genetic tests and matches the categories to preset solutions. Thus, it is difficult to determine the accurate learning tendency of a subject, and there are limitations in recommending appropriate educational programs accordingly.

### RELATED ART DOCUMENTS

(Patent Document 1) Korean Patent No. 10-2073195

### DISCLOSURE

### Technical Problem

The present invention is directed to providing a system for determining learning tendency information on the basis of genetic test information.

The present invention is also directed to providing a method of determining learning tendency information on the basis of genetic test information.

### Technical Solution

According to an aspect of the present invention, there is provided a system for determining learning tendency information on the basis of genetic test information, the system including a basic information generator configured to receive information on a subject and generate basic information, a genetic test information generator configured to generate genetic information by analyzing a genetic sample of the subject, and a learning tendency information determiner configured to generate cognitive-category learning tendency information on the basis of the genetic information, generate noncognitive-category learning tendency information on the basis of the genetic information and the basic information, and generate learning tendency information by synthesizing the cognitive-category learning tendency information and the noncognitive-category learning tendency information.

The system may further include a consulting part. The system may further include an educational program provision part configured to provide educational program information such that the consulting part may use the learning tendency information to match an appropriate educational program in the educational program information with the subject, or may further include a customized educational program provision part such that the consulting part may use the learning tendency information to generate an appropriate customized educational program for the subject.

The consulting part may provide a report including the learning tendency information to the subject or an educational program provider. The report may further include the basic information.

The system may further include a learning tendency information updater configured to receive the learning tendency information and then update the learning tendency information by incorporating learning achievement of the subject into the learning tendency information.

In generating the cognitive-category learning tendency information on the basis of the genetic information, the learning tendency information determiner may generate cognitive categories by subcategorizing cognitive tendencies that affect learning, crawl papers on genes that affect each of the cognitive categories, select a gene in accordance with reliability and an impact factor of each paper to give a weight to the gene, determine relationships between genes and learning tendency information of each of the cognitive categories, and generate learning tendency information of each of the cognitive categories using the genetic information on the basis of the relationships.

In generating the noncognitive-category learning tendency information on the basis of the genetic information and the basic information, the learning tendency information determiner may generate noncognitive categories by subcategorizing noncognitive tendencies that affect learning, crawl papers on genes that affect each of the noncognitive categories, select a gene in accordance with reliability and an impact factor of each paper to give a weight to the gene, determine relationships between genes and learning tendency information of each of the noncognitive categories, and generate learning tendency information of each of the noncognitive categories resulting from genetic factors on the basis of the relationships using the genetic information. The learning tendency information determiner may generate learning tendency information of each noncognitive category resulting from environmental factors using the basic information and generate the noncognitive-category learning tendency information by synthesizing learning tendency information of each noncognitive category resulting from the genetic factors and learning tendency information of each noncognitive category resulting from the environmental factors.

The learning tendency information determiner may use an artificial intelligence (AI)-based judgment algorithm to generate the learning tendency information by synthesizing the cognitive-category learning tendency information and the noncognitive-category learning tendency information, and the judgment algorithm may use genetic information and basic information of a plurality of subjects to classify a combination of the cognitive-category learning tendency information and the noncognitive-category learning tendency information into a type, may use learning achievement information included in the basic information to analyze differences in type between subjects with the same achievement and analyze differences in achievement between subjects with similar types of cognitive-category learning tendency information. The judgment algorithm may analyze differences in achievement between subjects with similar types of noncognitive category learning tendency information, analyze a reinforcement condition of the noncognitive category learning tendency information using learning environment information included in the basic information, and use correlations between the learning achievement information and the cognitive-category learning tendency information and the noncognitive-category learning tendency information.

According to another aspect of the present invention, there is provided a method of determining learning tendency information on the basis of genetic test information, the method including a genetic sampling operation of collecting a genetic sample of a subject, a survey information input operation of inputting information on the subject, an operation of generating genetic information by analyzing the genetic sample, an operation of generating basic information using the survey information, an operation of generating cognitive-category learning tendency information on the basis of the genetic information, an operation of generating noncognitive-category learning tendency information on the basis of the genetic information and the basic information, and an operation of generating learning tendency information by synthesizing the cognitive-category learning tendency information and the noncognitive-category learning tendency information.

The method may further include an educational program information input operation of inputting educational program information of an educational program provider and an educational program matching operation of matching an appropriate educational program in the educational program information with the subject. The method may further include a customized program generation operation of generating an appropriate customized educational program for the subject using the learning tendency information.

The method may further include an operation of providing a report including the learning tendency information to the subject or an educational program provider.

The report may further include the basic information and the survey information.

The method may further include a learning tendency information update operation of receiving the learning tendency information and then updating the learning tendency information by incorporating learning achievement of the subject into the learning tendency information.

The operation of generating the cognitive-category learning tendency information on the basis of the genetic information may include generating cognitive categories by subcategorizing cognitive tendencies that affect learning, crawling papers on genes that affect each of the cognitive categories, selecting a gene in accordance with reliability and an impact factor of each paper to give a weight to the gene, determining relationships between genes and learning tendency information of each of the cognitive categories, and generating learning tendency information of each of the cognitive categories using the genetic information on the basis of the relationships.

The operation of generating the noncognitive-category learning tendency information on the basis of the genetic information and the basic information may include generating noncognitive categories by subcategorizing noncognitive tendencies that affect learning, crawling papers on genes that affect each of the noncognitive categories, selecting a gene in accordance with reliability and an impact factor of each paper to give a weight to the gene, determining relationships between genes and learning tendency information of each of the noncognitive categories, and generating learning tendency information of each of the noncognitive categories resulting from genetic factors on the basis of the relationships using the genetic information. The basic information may be used to generate learning tendency information of each noncognitive category resulting from environmental factors. The noncognitive-category learning tendency information may be generated by synthesizing learning tendency information of each noncognitive category resulting from the genetic factors and learning tendency information of each noncognitive category resulting from the environmental factors.

In the operation of generating the learning tendency information by synthesizing the cognitive-category learning tendency information and the noncognitive-category learning tendency information, an AI-based judgment algorithm may be used to generate the learning tendency information. The judgment algorithm may use genetic information and basic information of a plurality of subjects to classify a combination of the cognitive-category learning tendency information and the noncognitive-category learning tendency information into a type, may use learning achievement information included in the basic information to analyze differences in type between subjects with the same achievement, analyze differences in achievement between subjects with similar types of cognitive-category learning tendency information, and analyze differences in achievement between subjects with similar types of noncognitive category learning tendency information, may use learning environment information included in the basic information to analyze a reinforcement condition of the noncognitive category learning tendency information, and may use correlations between the learning achievement information and the cognitive-category learning tendency information and the noncognitive-category learning tendency information.

The method may further include an operation of updating the learning tendency information.

### Advantageous Effects

According to exemplary embodiments of the present invention, it is possible to generate cognitive-category learning tendency information and noncognitive-category learning tendency information on the basis of a subject's genetic information and basic information, synthesize the cognitive-category learning tendency information and the noncognitive-category learning tendency information, identify innate biological disposition (genetic factors) and environments, and separately acquire learning tendency information of cognitive and noncognitive categories. Accordingly, learning tendency information can be determined more accurately, and thus an effective educational program can be provided.

Effects of the present invention are not limited to that described above, and may be variously expanded without departing from the spirit and scope of the present invention.

### DESCRIPTION OF DRAWINGS

FIG. 1 is a schematic block diagram of a system for determining learning tendency information on the basis of genetic test information according to an exemplary embodiment of the present invention.

FIG. 2 is a flowchart illustrating a method of determining learning tendency information on the basis of genetic test information according to an exemplary embodiment of the present invention.

FIG. 3 is a detailed flowchart of a learning tendency information generation operation of FIG. 2.

FIG. 4 is a flowchart illustrating a consulting operation of the method of determining learning tendency information on the basis of genetic test information according to the exemplary embodiment of the present invention.

### MODES OF THE INVENTION

Hereinafter, exemplary embodiments of the present invention will be described in detail with reference to the accompanying drawings.

The present invention may be modified in various ways and have various embodiments, and specific embodiments will be illustrated in the drawings and described in detail. However, this is not intended to limit the present invention to specific disclosed forms, and it is to be understood that the present invention includes all modifications, equivalents, and substitutions within the spirit and technical scope of the present invention.

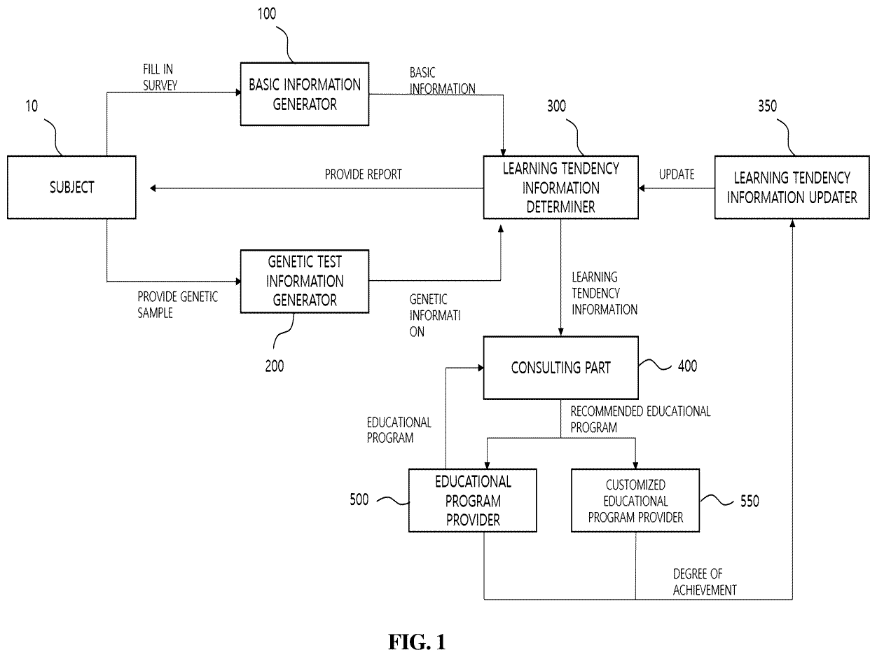

FIG. 1 is a schematic block diagram of a system for determining learning tendency information on the basis of genetic test information according to an exemplary embodiment of the present invention.

Referring to FIG. 1, the system for determining learning tendency information on the basis of genetic test information may include a basic information generator 100, a genetic test information generator 200, a learning tendency information determiner 300, a learning tendency information updater 350, a consulting part 400, an educational program provision part 500, and a customized educational program provision part 550.

The basic information generator 100 may receive information on a subject and generate basic information.

The genetic test information generator 200 may analyze a genetic sample of the subject and generate genetic information.

The learning tendency information determiner 300 may generate learning tendency information. The learning tendency information determiner 300 may generate cognitive-category learning tendency information on the basis of the genetic information or the genetic information and the basic information, generate noncognitive-category learning tendency information on the basis of the genetic information and the basic information, and generate the learning tendency information by synthesizing the cognitive-category learning tendency information and the noncognitive-category learning tendency information.

Specifically, the learning tendency information determiner 300 may generate cognitive-category learning tendency information on the basis of the genetic information. The learning tendency information determiner 300 may generate cognitive categories by subcategorizing cognitive tendencies that affect learning, crawl papers on genes that affect each of the cognitive categories, select a gene in accordance with reliability and an impact factor of each paper to give a weight to the gene, determine relationships between genes and learning tendency information of each of the cognitive categories, and generate learning tendency information of each of the cognitive categories using the genetic information on the basis of the relationships.

In addition, the learning tendency information determiner 300 may generate noncognitive-category learning tendency information on the basis of the genetic information and the basic information. The learning tendency information determiner 300 may generate noncognitive categories by subcategorizing noncognitive tendencies that affect learning, crawl papers on genes that affect each of the noncognitive categories, select a gene in accordance with reliability and an impact factor of each paper to give a weight to the gene, determine relationships between genes and learning tendency information of each of the noncognitive categories, and generate learning tendency information of each of the noncognitive categories resulting from genetic factors using the genetic information on the basis of the relationships.

The learning tendency information determiner 300 may use an artificial intelligence (AI)-based judgment algorithm to generate the learning tendency information by synthesizing the cognitive-category learning tendency information and the noncognitive-category learning tendency information.

The judgment algorithm may use genetic information and basic information of a plurality of subjects to classify a combination of the cognitive-category learning tendency information and the noncognitive-category learning tendency information into a type, may use learning achievement information included in the basic information to analyze differences in type between subjects with the same achievement, analyze differences in achievement between subjects with similar types of cognitive-category learning tendency information, and analyze differences in achievement between subjects with similar types of noncognitive category learning tendency information, may use learning environment information included in the basic information to analyze a reinforcement condition of the noncognitive category learning tendency information, and may use correlations between the learning achievement information and the cognitive-category learning tendency information and the noncognitive-category learning tendency information to generate the learning tendency information.

For example, the judgment algorithm may be an AI-based judgment algorithm. The judgment algorithm identifies correlations between items of cognitive-category learning tendency information and learning achievement and correlations between items of noncognitive-category learning tendency information and learning achievement and gives weights to items that affect the learning achievement overall on the basis of the correlations, preparing criteria to determine learning tendencies.

In addition, the judgment algorithm may select and recommend educational programs (lectures) for reinforcing the strengths of each of the items and complementing the weaknesses on the basis of the criteria, may track and crawl actual data (learning achievement, learning attitude, learning environments, and the like) while a user takes the recommended educational programs, and may compare the crawled data with learning tendency judgment data to analyze and verify the differences. Using this process, the AI-based judgment algorithm may be continuously improved by modifying and complementing weights in accordance with the degree to which each item affects learning achievement. After receiving the learning tendency information, the learning tendency information updater 350 may update the learning tendency information by incorporating learning achievement of the subject into the learning tendency information. This update may reflect information on the degree of achievement that is generated to reflect the learning achievement of the subject by an institution which provides the educational program to be described below.

The consulting part 400 may receive the learning tendency information and provide a report including the learning tendency information to the subject or an educational program provider. Whether genes related to learning categories (cognitive, noncognitive, multiple intelligence, brain science, and the like) have mutated may be analyzed as genetic test result data to provide a report in which the subject's genetic capabilities are identified, and only desired categories may be selected to provide a customized report. In other words, the report may provide information combined with basic information, survey information, and genetic learning tendency information to the subject or the educational program provider.

The educational program provision part 500 may provide information on providable educational programs to the consulting part 400, and the consulting part 400 may use the learning tendency information to match an appropriate educational program in the information on the educational programs with the subject.

When the consulting part 400 generates a customized educational program for the subject using the learning achievement information, the customized educational program provision part 550 may provide the recommended educational program to the subject in a customized manner.

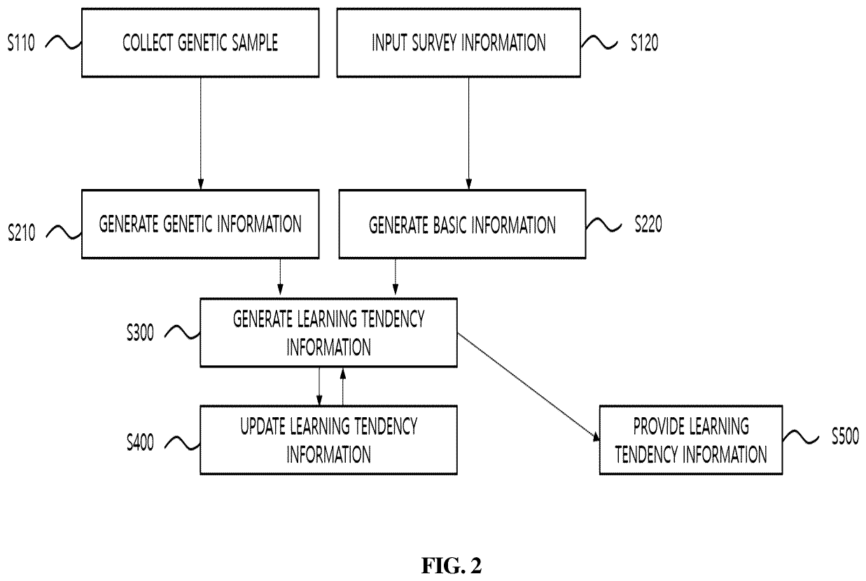

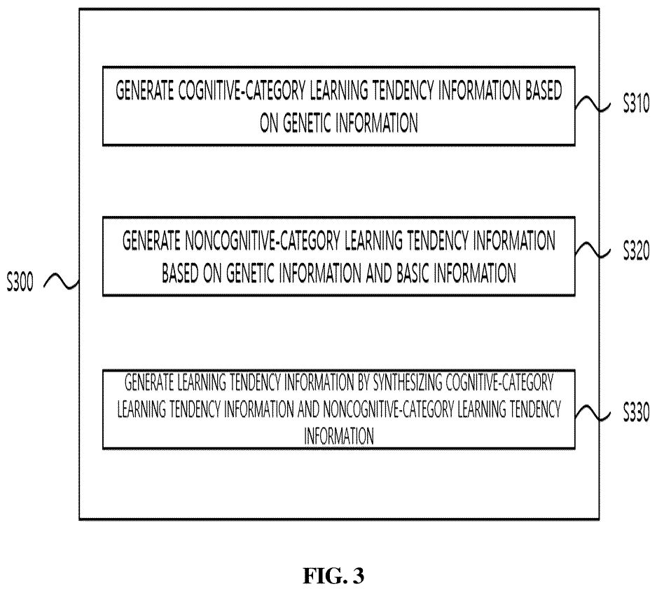

FIG. 2 is a flowchart illustrating a method of determining learning tendency information on the basis of genetic test information according to an exemplary embodiment of the present invention. FIG. 3 is a detailed flowchart of a learning tendency information generation operation of FIG. 2.

Referring to FIGS. 2 and 3, the method of determining learning tendency information on the basis of genetic test information may include a genetic sampling operation S110, a survey information input operation S120, a genetic information generation operation S210, a basic information generation operation S220, a learning tendency information generation operation S300, a learning tendency information update operation S400, and a learning tendency information provision operation S500.

In the genetic sampling operation S110, a bio-sample is collected from a subject. The bio-sample is a biological sample containing the genetic information of the subject. The biological sample may be in the form of a biological fluid. Detailed examples of the bio-sample may be saliva, a blood sample, a whole blood sample, and the like.

The survey information input operation S120, the subject or a guardian may input various information on the subject. When a questionnaire is provided through a mobile or Internet app, the web, or the like, various survey information may be input as responses to the questionnaire. For example, the survey information may be the subject's personal information, such as age, gender, family, and the like, physical capabilities, degree of exercise, learning environment, such as a learning time and the like, learning achievement, and the like.

In addition, in the survey information input operation S120, answers may be input to a questionnaire on noncognitive categories related to a living environment, personality, and disposition for generating noncognitive-category learning tendency information to be described below.

In the genetic information generation operation S210, a genetic test of the collected bio-sample may be performed through a genetic testing device to generate genetic information.

In the basic information generation operation S220, basic information of the subject may be generated from the survey information. The basic information may include various items related to cognitive categories and noncognitive categories. For example, the basic information may include objective indicators of the user's personal information, physical capabilities, learning capabilities, resilience, Big5 personality, multiple intelligence, and the like. Big5 is a psychological model of personality that describes personality in terms of five independent factors, neuroticism, extraversion, openness, agreeableness, and conscientiousness.

In the learning tendency information generation operation S300, cognitive-category learning tendency information may be generated on the basis of the genetic information, noncognitive-category learning tendency information may be generated on the basis of the genetic information and the basic information, and the cognitive-category learning tendency information and the noncognitive-category learning tendency information may be synthesized to generate learning tendency information.

Specifically, the learning tendency information generation operation S300 may include an operation S310 of generating cognitive-category learning tendency information on the basis of the genetic information, an operation S320 of generating noncognitive-category learning tendency information on the basis of the genetic information and the basic information, and an operation S330 of synthesizing the cognitive-category learning tendency information and the noncognitive-category learning tendency information to generate learning tendency information.

In the operation S310 of generating cognitive-category learning tendency information, cognitive tendencies that affect learning may be subcategorized, and papers on genes that affect each subcategory may be crawled. Genes may be selected in accordance with the reliability levels (level 2 of evidence or higher) and impact factors of the crawled papers to give weights to the genes such that the cognitive-category learning tendency information may be generated on the basis of the genetic information. Items corresponding to the cognitive-category may include, but are not limited to, comprehension, thinking power, creativity, concentration, and the like.

Meanwhile, an algorithm for generating the cognitive-category learning tendency information may generate the cognitive-category learning tendency information using an AI model.

In the operation S320 of generating noncognitive-category learning tendency information on the basis of the genetic information and the basic information, noncognitive tendencies that affect learning may be subcategorized, and papers on genes that affect each subcategory may be crawled. Genes may be selected in accordance with the reliability levels (level 2 of evidence or higher) and impact factors of the crawled papers to give weights to the genes such that the noncognitive-category learning tendency information may be generated on the basis of the genetic information.

The basic information may be used to generate learning tendency information of each noncognitive-category resulting from environmental factors.

The noncognitive-category learning tendency information may be generated by synthesizing learning tendency information of each noncognitive category resulting from the genetic factors and learning tendency information of each noncognitive category resulting from the environmental factors.

The noncognitive categories include, but are not limited to, physical capabilities, such as endurance, stamina, attention, agility, and the like, mental traits, such as resilience, self-regulation, self-esteem, positivity, and the like, Big5, and the like.

In the operation S330 of synthesizing the cognitive-category learning tendency information and the noncognitive-category learning tendency information to generate learning tendency information, the cognitive-category learning tendency information and the noncognitive-category learning tendency information may be synthesized to generate learning tendency information.

In synthesizing the cognitive-category learning tendency information and the noncognitive-category learning tendency information to generate learning tendency information, an AI-based judgment algorithm may be used to generate the learning tendency information.

The judgment algorithm may use genetic information and basic information of a plurality of subjects to classify a combination of the cognitive-category learning tendency information and the noncognitive-category learning tendency information into a type, may use learning achievement information included in the basic information to analyze differences in type between subjects with the same achievement, analyze differences in achievement between subjects with similar types of cognitive-category learning tendency information, and analyze differences in achievement between subjects with similar types of noncognitive category learning tendency information, may use learning environment information included in the basic information to analyze a reinforcement condition of the noncognitive category learning tendency information, and may use correlations between the learning achievement information and the cognitive-category learning tendency information and the noncognitive-category learning tendency information to generate the learning tendency information.

For example, the judgment algorithm may be an AI-based judgment algorithm. The judgment algorithm identifies correlations between items of the cognitive-category learning tendency information and the learning achievement and correlations between items of the noncognitive-category learning tendency information and the learning achievement and gives weights to items that affect the learning achievement overall on the basis of the correlations, thus preparing criteria to determine learning tendencies. In the learning tendency information update operation S400, the learning tendency information is received and then updated to reflect the learning achievement of the subject. Specifically, the learning tendency information may be updated to compare, re-estimate, and re-recommend the learning achievement of the subject.

Here, the learning achievement and information related to the living environment may be continuously updated such that the trend of the subject's learning achievement can be checked. For example, as an achievement measure for learning and teaching methods, a trend in academic achievement may be observed through continuous updates of learning achievement information, such as tests, examination scores, and the like, information on creating an environment for fostering positive emotions or mental strength (grit, meditation, or exercise), desires to accomplish, such as competitive spirit and the like, learning attitudes, such as in-class behavior, concentration, and the like, and end-of-course assessment information, such as academic persistence and the like.

In the learning tendency information provision operation S500, a report including the learning tendency information may be provided to the subject or an educational program provider. Whether genes related to learning categories (cognitive, noncognitive, multiple intelligence, brain science, and the like) have mutated is analyzed as genetic test result data to provide a report in which the subject's genetic competency is identified, and only desired categories may be selected to provide a customized report. In other words, the report may provide information combined with basic information, survey information, and genetic learning tendency information to the subject or the educational program provider. In addition, the report may also provide basic information and tips on the description of the subject's genetic characteristics related to learning tendency information, a reinforcement method for genetic characteristics, a complementary method, and the like in the form of text or a video clip.

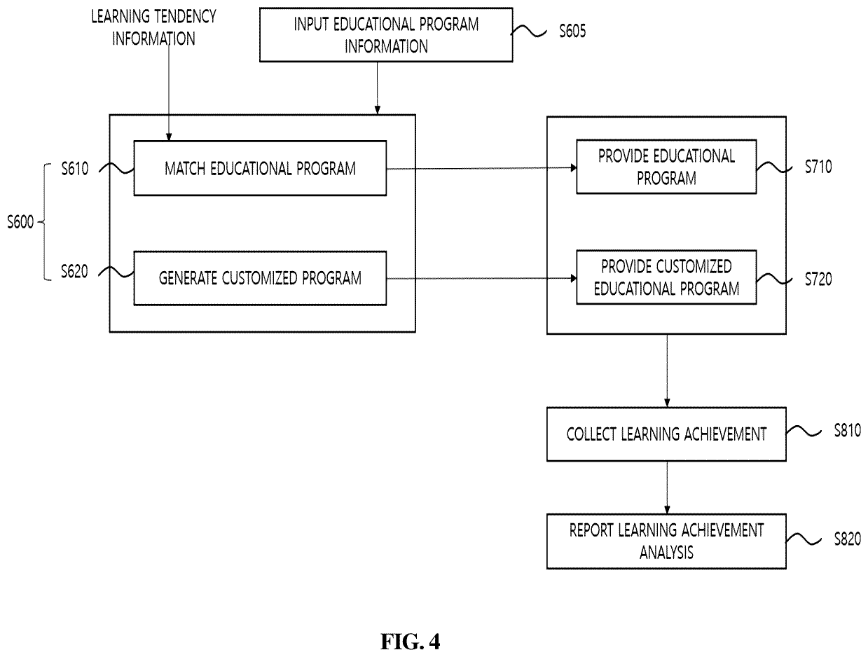

FIG. 4 is a flowchart illustrating a consulting operation of the method of determining learning tendency information on the basis of genetic test information according to the exemplary embodiment of the present invention.

Referring to FIG. 4, the method of determining learning tendency information on the basis of genetic test information may further include a consulting operation S600, an educational program provision operation S710, a customized educational program provision operation S720, a learning achievement collection operation S810, and a learning achievement analysis report provision operation S820.

The consulting operation S600 may further include an educational program information input operation S605, an educational program matching operation S610, and a customized program generation operation S620.

In the educational program information input operation S605, a providable educational program may be input. In the educational program matching operation S610, the learning tendency information may be used to match an appropriate educational program in the educational program information to the subject. In the customized program generation operation S620, the learning tendency information may be used to generate an appropriate customized educational program for the individual subject.

For example, educational programs (lectures) for reinforcing the strengths of each of the items in the cognitive-or noncognitive-category learning tendency information and complementing the weaknesses may be selected and recommended, and actual data (learning achievement, learning attitude, learning environments, and the like) may be tracked and crawled while the user takes the recommended educational programs, and may be compared with learning tendency judgment data to analyze and verify the differences.

The educational program or the customized educational program may be, for example, AI-based customized information of associated study institutes for maximizing learning effects or may include educational programs for maximizing learning effects through an AI algorithm.

In the educational program provision operation S710 and the customized educational program provision operation S720, a corresponding educational institution may provide the educational program or the customized educational program to the subject.

In the learning achievement collection operation S810, the subject's learning activities may be monitored to measure the degree of achievement of a type-specific learning method. The measured degree of achievement may be utilized to train or update the judgment algorithm, and an optimal learning method may be recommended in accordance with the genetic learning tendency. For example, the AI-based judgment algorithm may be continuously updated by correcting or complementing weights in accordance with the degree to which each item affects learning achievement based on the judgement algorithm. The degree of achievement may include institution-specific learning method/type data or teacher-specific learning method/type data. The degree of achievement may include information such as a current class enrollment status, progress, attendance, learning record information, and the like.

In the learning achievement analysis report provision operation S820, a learning achievement analysis report may be provided to the subject or the educational program provider on the basis of the degree-of-achievement information.

For example, to establish and implement a customized educational direction for the subject and manage learning achievement in an educational institution, institute, school, or the like in which the subject has agreed to provide information, information of each learning tendency result sheet (degree of achievement) may be summarized, excerpted, and grouped and provided to the institute and a teacher.

For example, the educational program and the customized educational program may be provided to an institute teacher, and the achievement of the solution may be compared, categorized, summarized, excerpted, and grouped such that the information may be provided to a manager and the teacher for the purpose of learning management.

The information may be provided or input using a personal computer (PC) web system, and the subject's current class enrollment status, class progress, attendance, learning records and data, and the like may be recorded and provided to the subject and a consultant.

According to the present embodiment, a subject or a subject's guardian, an educational program provider, and the like can be continuously provided with information about a recommended educational program and the subject's class enrollment status, progress, attendance, learning record information, and the like, and the subject can be provided with continuous feedback on learning consulting (explanation of a genetic analysis report and aspects related to improving the child's learning ability such as the child's psychological state, educational program, and the like).

According to exemplary embodiments of the present invention, it is possible to generate cognitive-category learning tendency information and noncognitive-category learning tendency information on the basis of a subject's genetic information and basic information, synthesize the cognitive-category learning tendency information and the noncognitive-category learning tendency information, identify innate biological disposition (genetic factors) and environments, and separately acquire learning tendency information of cognitive and noncognitive categories. Accordingly, learning tendency information can be determined more accurately, and thus an effective educational program can be provided.

Although the present invention has been described above with reference to exemplary embodiments, those of ordinary skill in the art can make various modifications and alterations of the present invention without departing from the spirit and scope of the present invention described in the following claims.

### DESCRIPTION OF SIGNS

10: subject 100: basic information generator
200: genetic test information generator 300: learning tendency information determiner
350: learning tendency information updater 400: consulting part
500: educational program provision part 550: customized educational program provision part

1. A system for determining learning tendency information on the basis of genetic test information, the system comprising:
a basic information generator configured to receive information on a subject and generate basic information;
a genetic test information generator configured to generate genetic information by analyzing a genetic sample of the subject; and
a learning tendency information determiner configured to generate cognitive-category learning tendency information on the basis of the genetic information, generate noncognitive-category learning tendency information on the basis of the genetic information and the basic information, and generate learning tendency information by synthesizing the cognitive-category learning tendency information and the noncognitive-category learning tendency information,
wherein the cognitive-category learning tendency information includes information on one or more of comprehension, thinking power, creativity, and concentration,
the noncognitive-category learning tendency information includes information on one or more of endurance, stamina, attention, and agility,
the learning tendency information determiner generates cognitive categories by subcategorizing cognitive tendencies that affect learning and generates the cognitive-category learning tendency information using the genetic information,
generates noncognitive categories by subcategorizing noncognitive tendencies that affect learning, generates noncognitive-category learning tendency information resulting from genetic factors, and generates noncognitive-category learning tendency information resulting from environmental factors, and
generates the noncognitive-category learning tendency information by synthesizing learning tendency information of each noncognitive category resulting from the genetic factors and learning tendency information of each noncognitive category resulting from the environmental factors,
the learning tendency information determiner uses an artificial intelligence (AI)-based judgment algorithm to generate the learning tendency information by synthesizing the cognitive-category learning tendency information and the noncognitive-category learning tendency information, and
the judgment algorithm uses genetic information and basic information of a plurality of subjects to classify a combination of the cognitive-category learning tendency information and the noncognitive-category learning tendency information into a type,
uses learning achievement information included in the basic information to analyze differences in type between subjects with the same achievement, analyze differences in achievement between subjects with similar types of cognitive-category learning tendency information, and analyze differences in achievement between subjects with similar types of noncognitive category learning tendency information, and
uses learning environment information included in the basic information to analyze a reinforcement condition of the noncognitive category learning tendency information, and uses correlations between the learning achievement information and the cognitive-category learning tendency information and the noncognitive-category learning tendency information.

2. The system of claim 1, further comprising:
a consulting part; and
an educational program provision part configured to provide educational program information such that the consulting part uses the learning tendency information to match an appropriate educational program in the educational program information with the subject, or
a customized educational program provision part such that the consulting part uses the learning tendency information to generate an appropriate customized educational program for the subject.

3. The system of claim 2, wherein the consulting part provides a report including the learning tendency information to the subject or an educational program provider, and
the report further includes the basic information.

4. The system of claim 3, further comprising a learning tendency information updater configured to receive the learning tendency information and then update the learning tendency information by incorporating learning achievement of the subject into the learning tendency information and comparing the learning achievement with the learning tendency information.

5. The system of claim 1, wherein, in generating the cognitive-category learning tendency information on the basis of the genetic information, the learning tendency information determiner crawls papers on genes that affect each of the cognitive categories, selects a gene in accordance with reliability and an impact factor of each paper to give a weight to the gene, and determines relationships between genes and learning tendency information of each of the cognitive categories and a degree of influence of the genes in the learning tendency information.

6. The system of claim 5, wherein, in generating the noncognitive-category learning tendency information on the basis of the genetic information and the basic information, the learning tendency information determiner crawls papers on genes that affect each of the noncognitive categories, selects a gene in accordance with reliability and an impact factor of each paper to give a weight to the gene, and determines relationships between genes and learning tendency information of each of the noncognitive categories and a degree of influence of the genes in the learning tendency information.
